(19) United States
(12) Patent Application Publication
(10) Pub. No.: US 2026/0174995 A1
(43) Pub. Date: Jun. 25, 2026

(54) ANTISEPTIC CATHETER FIXATION DEVICE

(71) Applicant: SHEBA IMPACT LTD., Ramat Gan (IL)

(72) Inventors: Yonatan GARGI, Ganey Tikva (IL); Amir COHEN, Ramat Gan (IL); Valery NUDELMAN, Rishon Lezion (IL)

(21) Appl. No.: 19/126,493

(22) PCT Filed: Nov. 6, 2023
(86) PCT No.: PCT/IL2023/051138

§ 371 Date: May 1, 2025

(30) Foreign Application Priority Data
Nov. 6, 2022 (IL) 297985

(51) Int. Cl. A61M 25/02; A61M 25/00

(52) U.S. Cl. CPC A61M 25/02; A61M 2025/0056; A61M 2025/0246; A61M 2025/0266; A61M 2025/028; A61M 2205/13

(57) ABSTRACT

The present disclosure relates to antiseptic catheter fixation devices and dressings. In an example, an antiseptic catheter fixation device can include a base and a cover removably connectable to the. The base can include a hub chamber with an antiseptic gel therein, and a catheter insertion cavity covered at a first end thereof by a membrane having a catheter insertion cavity. The cover can include an antiseptic gel therein. The catheter insertion cavity can be configured to receive antiseptic liquid flowing therethrough, or to accommodate an antiseptic gel therein.

### CROSS-REFERENCE TO RELATED APPLICATIONS

This application claims the benefit of Israeli Patent Application No. 297985, filed Nov. 6, 2022, which is incorporated by reference herein.

### FIELD

The present invention relates generally to a catheter fixation device. More specifically, the present invention relates to an antiseptic catheter fixation device.

### BACKGROUND

Central catheter related blood stream infection (CRBSI) is one of the most serious complications in hospitalized patients, leading to increased hospitalization, intensive care admissions, extensive antibiotic treatment and mortality. It is also associated with profound costs.

In short-term central catheters (e.g., of less than 14 days), bacterial biofilm formation, originating from the central catheter skin insertion point, is a leading cause of such infection. Several methods for decreasing the incidence of central catheter infection have been proposed, including sterile techniques for central line insertion, the use of coated catheters and the use of chlorhexidine dressing. There is also evidence of reduced incidence of central catheter infections by using suture-less fixation methods.

Most of the known catheter fixation methods are based on sutures.

Accordingly, there is a need for a catheter fixation device that provides sterile conditions.

### SUMMARY

According to some aspects of the disclosure, there is provided an antiseptic catheter fixation device comprising a base and a cover removably connectable to the base. The base comprises a hub chamber, a catheter insertion cavity, and a membrane covering a first end of the catheter insertion cavity, wherein the membrane defines a catheter insertion orifice.

In some examples, the device further comprises an antiseptic gel residing inside the hub chamber.

In some examples, the device further comprises an antiseptic gel residing inside the cover.

In some examples, the device further comprises a cavity sidewall surrounding the catheter insertion cavity.

In some examples, the base comprises an attachment surface, opposite to the side of the hub chamber.

In some examples, the membrane comprises a bump defining an angled surface extending towards the catheter insertion cavity.

In some examples, the base is separatable into two parts that form two inner separatable sides facing each other.

In some examples, the device further comprises an antiseptic gel inside the catheter insertion cavity.

In some examples, the device further comprises at least two skin stretching elements extracted inwards into the catheter insertion cavity.

In some examples, each skin stretching element comprises a skin stretching wire, having a first wire portion embedded inside the antiseptic gel which is inside the catheter insertion cavity.

In some examples, the device further comprises an inlet tube and an outlet tube, both of which being in fluid communication with the catheter insertion cavity.

According to some aspects of the disclosure, there is provided a foldable antiseptic dressing comprising a foldable pad made from an antiseptic gel. The foldable pad comprises a folding portion and a catheter insertion strap. The folding portion comprises a thick support and a thinner strap. The catheter insertion strap comprises a recess for holding a catheter, wherein the catheter insertion strap is pivotally connected to the thinner strap.

In some examples, the dressing further comprises an adhesive material covering at least a portion of an attachment surface of the thick support, such that at a folded state, the adhesive material is configured to fixate the foldable antiseptic catheter fixation pad to a skin of a subject, such that the recess faces away from the skin of the subject when adhered thereto.

In some examples, the thickness of the catheter insertion strap is equal to or higher than a difference between the thicknesses of the thick support and the thinner strap.

The aspects of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the invention will become more apparent from the following detailed description, which proceeds with reference to the accompanying figures.

### BRIEF DESCRIPTION OF THE DRAWINGS

Some examples of the invention are described herein with reference to the accompanying figures. The description, together with the figures, makes apparent to a person having ordinary skill in the art how some examples may be practiced. The figures are for the purpose of illustrative description and no attempt is made to show structural details of an example in more detail than is necessary for a fundamental understanding of the invention. For the sake of clarity, some objects depicted in the figures are not to scale.

In the Figures:

FIGS. 1A-1B are illustrations of an antiseptic catheter fixation device according to some examples.

FIGS. 1C-1K are detailed illustrations of some parts included in the antiseptic catheter fixation device according to some examples.

FIGS. 2A-2C are illustrations of a separatable antiseptic catheter fixation device according to some examples.

FIG. 2D is an illustration of a separatable membrane for the separatable antiseptic catheter fixation device according to some examples.

FIGS. 3A-3D are illustrations of an antiseptic catheter fixation device according to some examples.

FIGS. 3E-3F are illustrations of exemplary skin stretching wires coupled to an antiseptic gel according to some examples.

FIGS. 4A-4E are illustrations of an antiseptic dressing for a central catheter according to some examples.

FIG. 5 is an image of the antiseptic dressing of FIGS. 4A-4E, providing antiseptic protection to a catheter inserted into a patient, according to some examples.

FIGS. 6A, 6B, and 6C show an image, tables, and a graph, respectively, summarizing an experiment demonstrating the increased rate of bacteria/yeasts growth under a hub of the central catheter according to some examples.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION

For purposes of this description, certain aspects, advantages, and novel features of the examples of this disclosure are described herein. The disclosed methods, devices, and dressings should not be construed as being limiting in any way. Instead, the present disclosure is directed toward all novel and nonobvious features and aspects of the various disclosed examples, alone and in various combinations and sub-combinations with one another. The methods, devices, and dressings are not limited to any specific aspect or feature or combination thereof, nor do the disclosed examples require that any one or more specific advantages be present, or problems be solved. The technologies from any example can be combined with the technologies described in any one or more of the other examples. In view of the many possible examples to which the principles of the disclosed technology may be applied, it should be recognized that the illustrated examples are only preferred examples and should not be taken as limiting the scope of the disclosed technology.

All features described herein are independent of one another and, except where structurally impossible, can be used in combination with any other feature described herein.

As used in this application and in the claims, the singular forms “a,” “an,” and “the” include the plural forms unless the context clearly dictates otherwise. Additionally, the terms “have” or “includes” means “comprises”. Further, the terms “coupled”, “connected”, and “attached”, as used herein, are interchangeable and generally mean physically, mechanically, chemically, magnetically, and/or electrically coupled or linked and does not exclude the presence of intermediate elements between the coupled or associated items absent specific contrary language. As used herein, “and/or” means “and” or “or”, as well as “and” and “or”.

Directions and other relative references may be used to facilitate discussion of the drawings and principles herein, but are not intended to be limiting. For example, certain terms may be used such as “inner,” “outer,” “upper,” “lower,” “inside,” “outside,”, “top,” “bottom,” “interior,” “exterior,” “left,” right,” and the like. Such terms are used, where applicable, to provide some clarity of description when dealing with relative relationships, particularly with respect to the illustrated examples. Such terms are not, however, intended to imply absolute relationships, positions, and/or orientations. For example, with respect to an object, an “upper” part can become a “lower” part simply by turning the object over. Nevertheless, it is still the same part and the object remains the same.

The term “plurality” or “plural” when used together with an element means two or more of the element. Directions and other relative references (e.g., inner and outer, upper and lower, above and below, left and right, and proximal and distal) may be used to facilitate discussion of the drawings and principles herein but are not intended to be limiting.

The terms “proximal” and “distal” are defined relative to the use position of a device or dressing. In general, the end of the device or dressing, when used in combination with a catheter, which is closest to the insertion point of the catheter through the skin of a subject, is the distal end, and the end of the device or dressing farthest from the catheter insertion point through the skin, is the proximal end. The term “proximal” when used with two spatially separated positions or parts of an object can be understood to mean closer to or oriented towards the proximal end of the device or dressing. The term “distal” when used with two spatially separated positions or parts of an object can be understood to mean closer to or oriented towards the distal end of the device or dressing.

Aspects of the disclosure may be directed to an antiseptic catheter fixation device. The device may solve three main problems in current days of catheter insertion: central catheter infections, catheter fixation, and bleeding prevention. Such an antiseptic catheter fixation device may allow a sterile insertion of a catheter, thus may reduce and even eliminating the incidence of central catheter infections. In some examples, the device may provide suture-less fixation that fits all kinds of short-term central catheters. Furthermore, currently, there is no known solution for insertion point bleeding besides applying a suture around the insertion point, and strangulating the insertion point. The device according to examples of the invention may provide stretching elements for stretching adjacent skin, thus reducing the bleeding.

As used herein, an antiseptic substance (e.g., gel, liquid, etc.) may be any substance (e.g., isopropyl alcohol or chlorhexidine) that destroys or inhibits the growth or action of microorganisms (e.g., bacteria) especially in or on living tissue (e.g., the skin or mucous membranes).

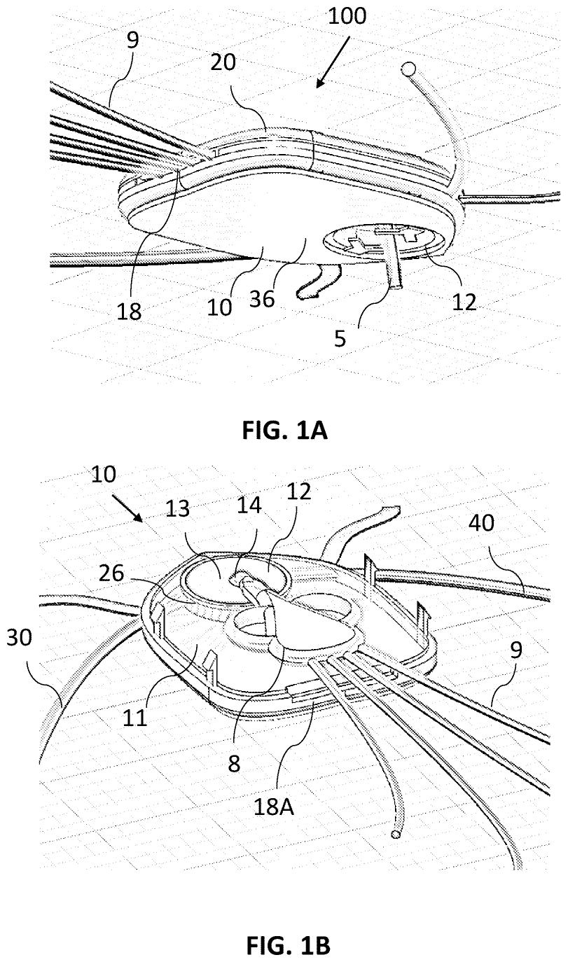

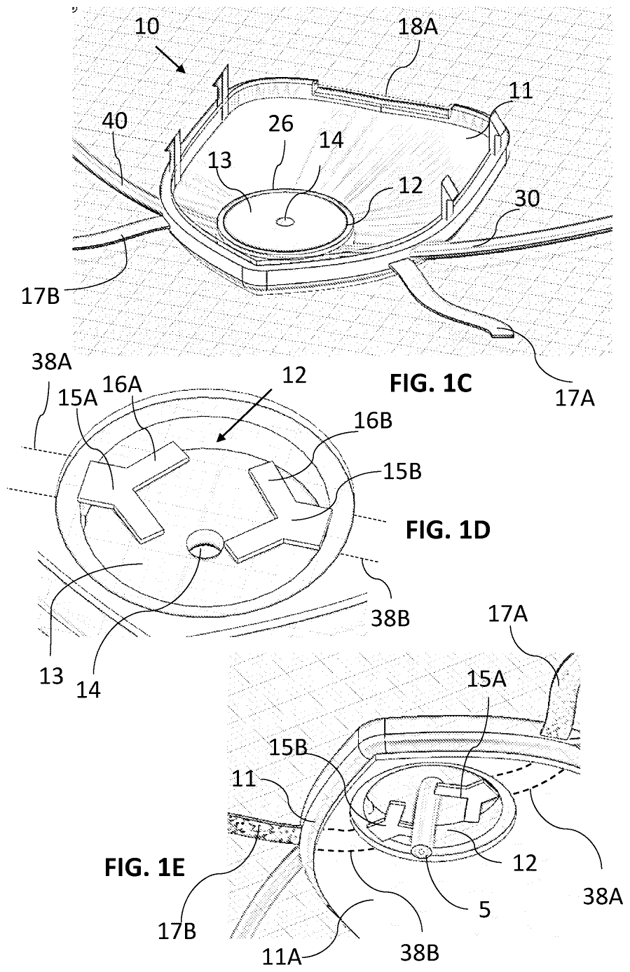

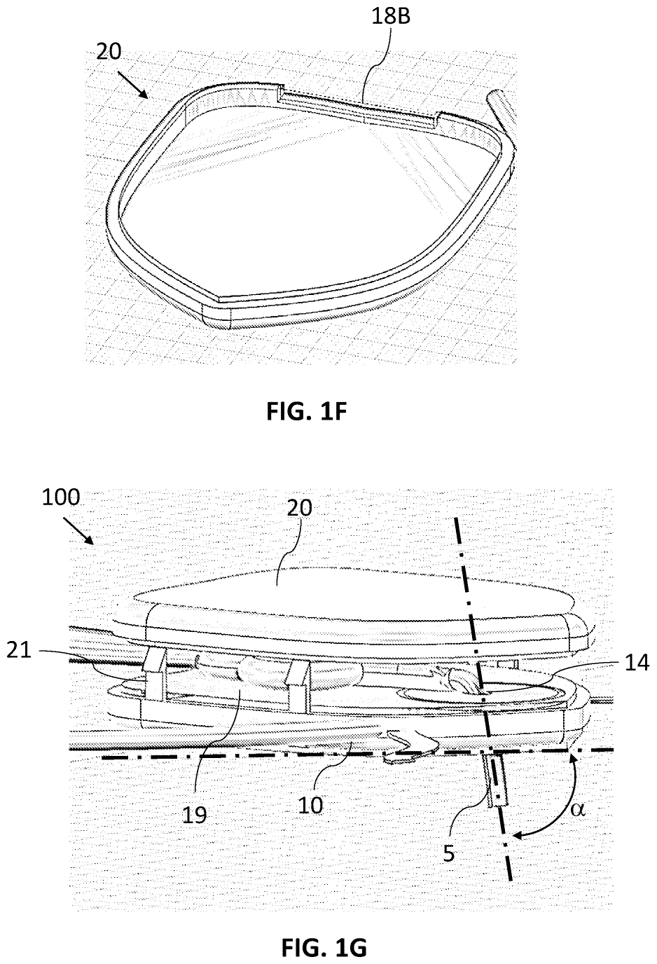

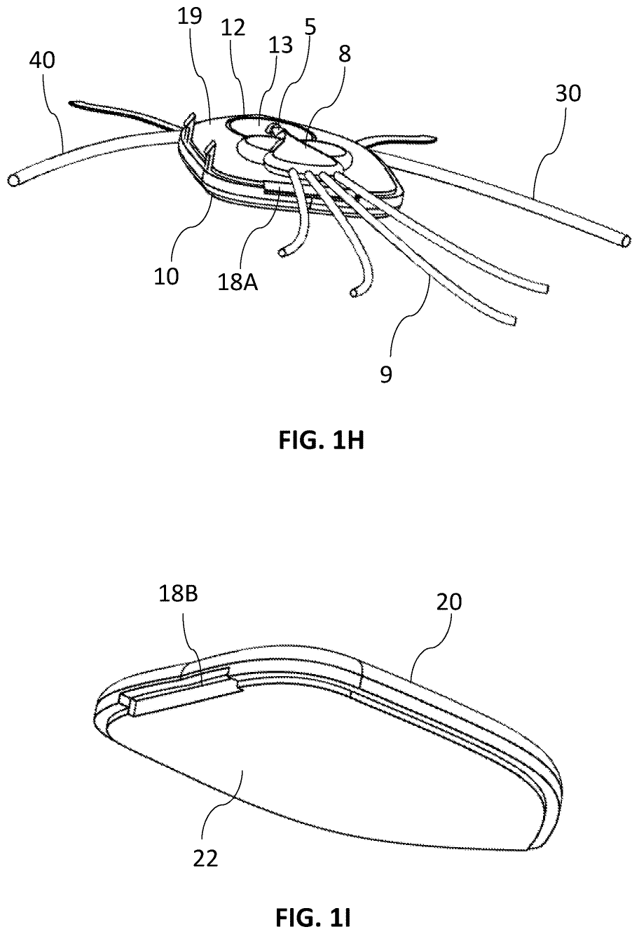

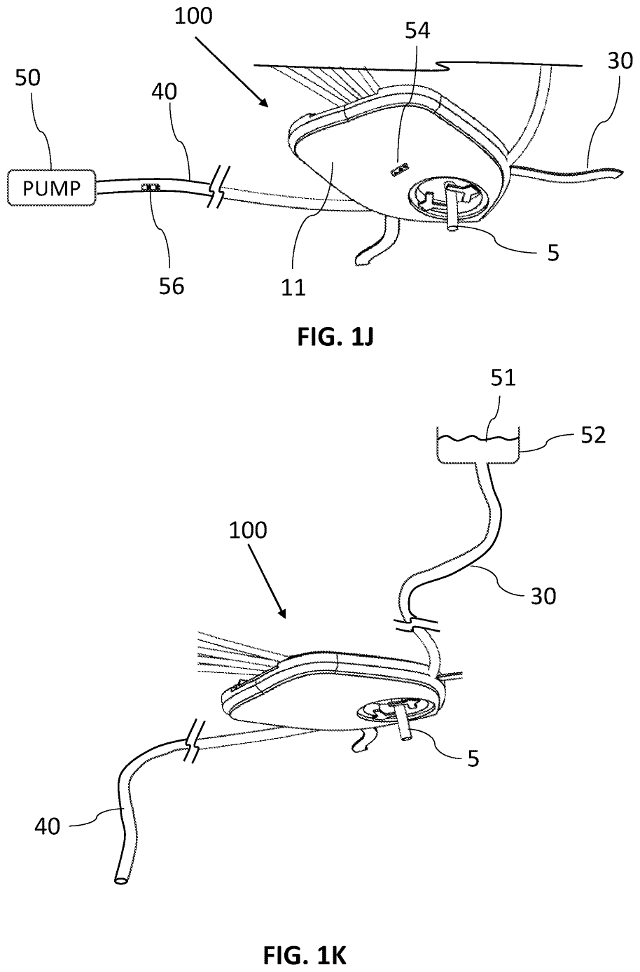

Reference is now made to FIGS. 1A and 1B which are illustrations of an antiseptic catheter fixation device according to some examples of the invention. An antiseptic catheter fixation device 100 may include a base 10 comprising a hub chamber 11 that can include antiseptic gel 19 (indicated, for example, in FIG. 1H), and a catheter insertion cavity 12 configured to be filled by antiseptic liquid 51 (indicated, for example, in FIG. 1I). A cavity sidewall 26 can surround the catheter insertion cavity 12 and separate between the catheter insertion cavity 12 and the hub chamber 11. Base 10 can be made from any suitable material, such as, medical-grade polymers, medical-grade alloys, and the like. The base 10 includes an attachment surface 36, opposite to the side of the hub chamber 11, configured to contact the skin of a patient into which a catheter 5 may be inserted.

In some examples, the attachment surface 36 is covered with an adhesive surface to be attached to a skin of a subject. The adhesive may be any medical-grade adhesive. In some examples, attachment surface 36 comprises at least one sticker (not shown) comprising an adhesive surface to be attached to a skin of a subject. The added adhesive may fixate the device and thus, also catheter 5 to the subject's body, ensuring a correct and fixed insertion of catheter 5 to the insertion point. In a nonlimiting example, high-grade biocompatible hydrophobic sticker may be added to the attachment surface 36 of base 10, to form a strong and durable fixation of device 100 to the skin surface. Such fixation may prevent any leak between catheter insertion cavity 12 and the external skin surface, and secure catheter 5 to its place. The terms “antiseptic catheter fixation device”, “catheter fixation device”, and “device”, as used herein, are interchangeable.

The catheter insertion cavity 12, located inside base 10, is covered at a first end by a membrane 13 comprising a catheter insertion orifice 14. In some examples, membrane 13 comprising catheter insertion orifice 14 is configured to direct catheter 5 to be inserted at an angle a (illustrated in FIG. 1G) of 30 to 90 degrees from the insertion side of catheter 5. The angle a can be defined between the catheter 5 and the attachment surface 36 or any surface parallel thereto, representing the surface of the patient's skin when the device 100 is laid thereover. This arrangement may prevent the accumulation of bacterial biofilm between the catheter and the subject's skin, by allowing a sufficient provision of antiseptic liquid flow to the surrounding of the catheter insertion point, as discussed herein below.

In some examples, membrane 13 may be made from any medical grade elastomer and may provide sealing to a catheter 5 inserted in catheter insertion orifice 14 and the escape of the antiseptic liquid 51. In a nonlimiting example, the membrane is made from silicone. In some examples, membrane 13 may centralize catheter 5 inside catheter insertion cavity 12, in a way that antiseptic liquid rinses contact catheter 5 line segment within the catheter insertion cavity 12 circumferentially. Membrane 13 may prevent leaks from the catheter insertion cavity 12. Orifice 14 may be adjustable to variable diameters of different short-term catheter types. The material of membrane 13 can be sufficiently flexible such that the orifice 14 can be enlarged when a larger diameter catheter 5 is passed therethrough, while sealing the membrane 13 around the catheter 5 passed through orifice 14. Thus, a single device 100 can be used with a variety of catheter 5 sizes.

Reference is now made to FIGS. 1C, 1D, 1E, 1F, 1G, 1H, 1I, 1J and 1K which are detailed illustrations of some parts included in the antiseptic catheter fixation device according to some examples.

In some examples, at least two (e.g., 2, 3, 4, etc.) skin stretching elements 15A and 15B may be extracted inwards into catheter insertion cavity 12, each connect to a corresponding stretching strap 17A and 17B. At least two skin-stretching elements 15A and 15B may be configured to stretch the skin in the vicinity of catheter 5 insertion point in order to avoid bleeding from the insertion point. In some examples, each skin stretching element 15A or 15B comprises an adhesive surface 16A and 16B to be attached to a skin of a subject. In some examples, each skin stretching element 15A or 15B is configured to move within catheter insertion cavity 12. In some examples, stretching elements 15A and 15B are configured to move radially, such as in a direction away from the catheter insertion orifice 14. Therefore, when a user pulls stretching straps 17A and 17B away from device 100, skin stretching elements 15A and 15B attached to the subject's skin, stretch the skin around the insertion point.

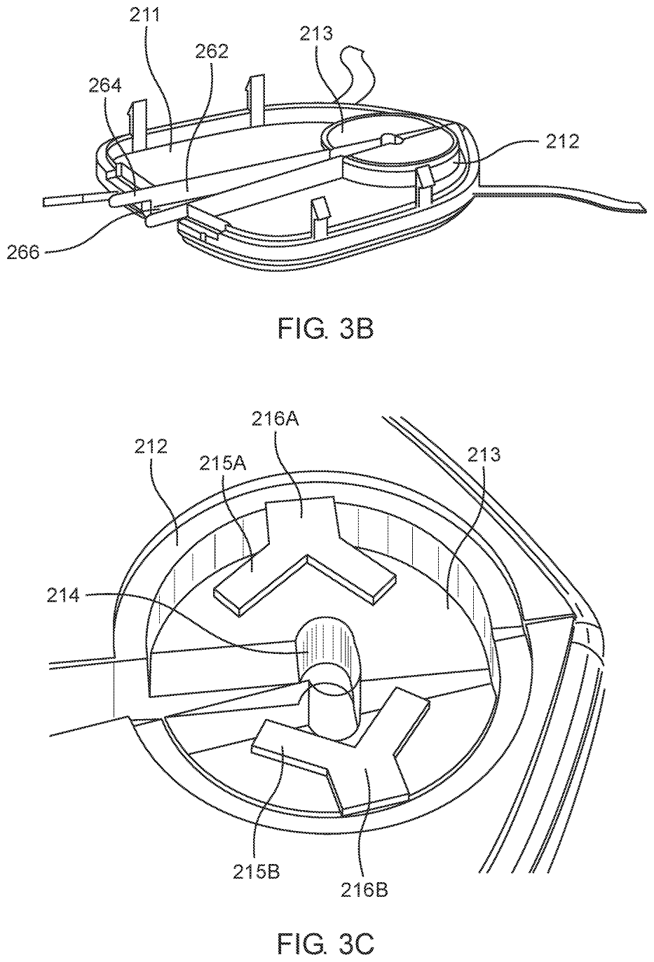

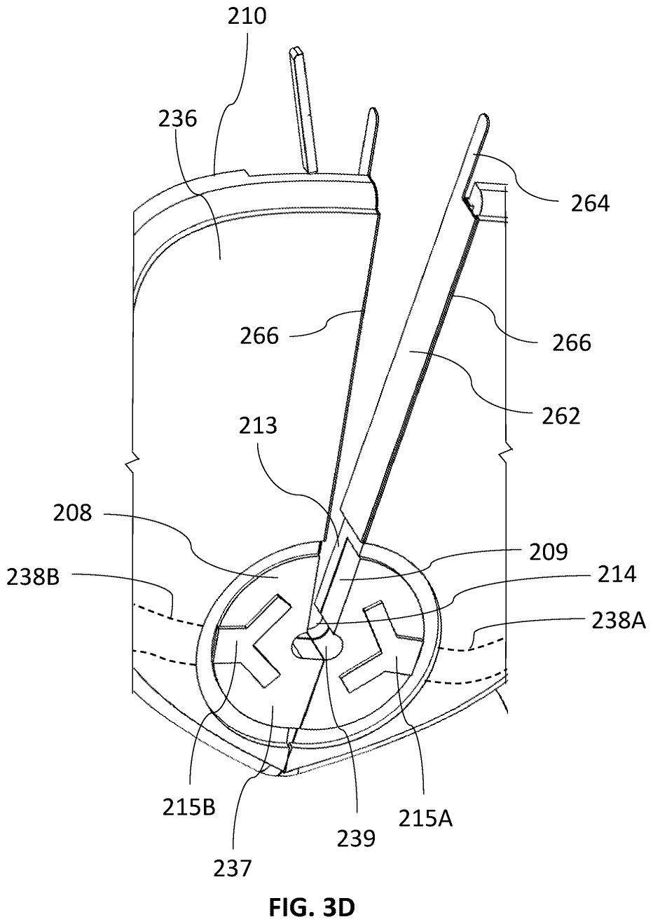

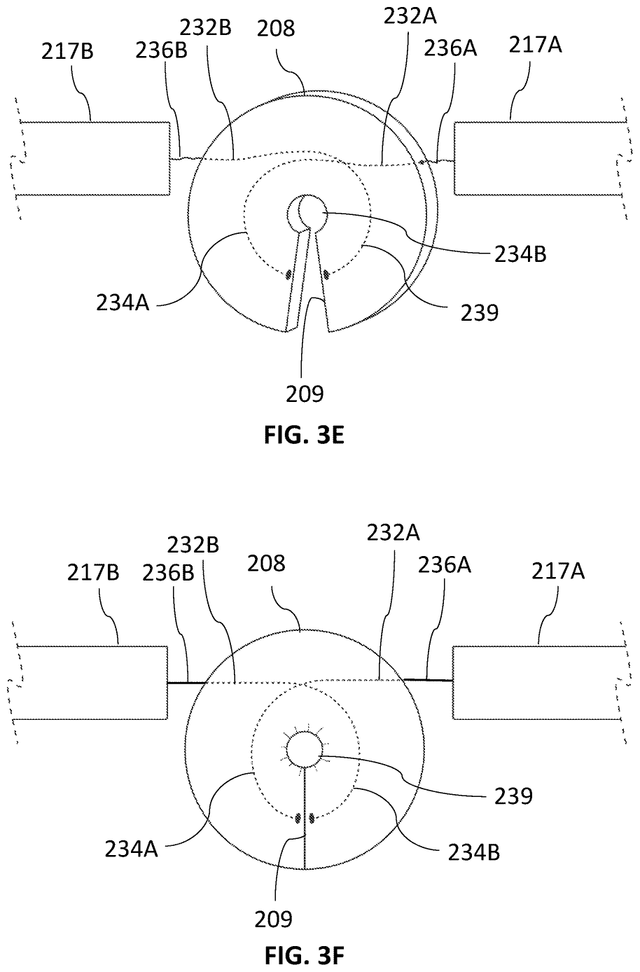

In the nonlimiting example illustrated in FIGS. 1D and 1E, the skin stretching elements 15A and 15B comprise V-shaped or fork-like end portion that can adhere to the skin from both sides of the catheter insertion point. The fork-like shape can increase the area of attachment to the patient's skin. It is to be understood that while a V-shaped end portion is illustrated, any other shape is contemplated, optionally having an inner enlarged surface relative to the main body (such as main bodies 216A and 216B illustrated for similarly shaped skin stretching elements 215A and 215B in FIG. 3C) of the element 15 extending towards the respective strap 17. Stretching two stretch straps 17A and 17B, connected to fork-like (or otherwise shaped) skin stretching elements 15A and 15B via thin tunnels 38A and 38B inside the base 10, pulls them away from each other laterally and causes stretching of the skin at the insertion point and thereby reduces insertion point bleeding.

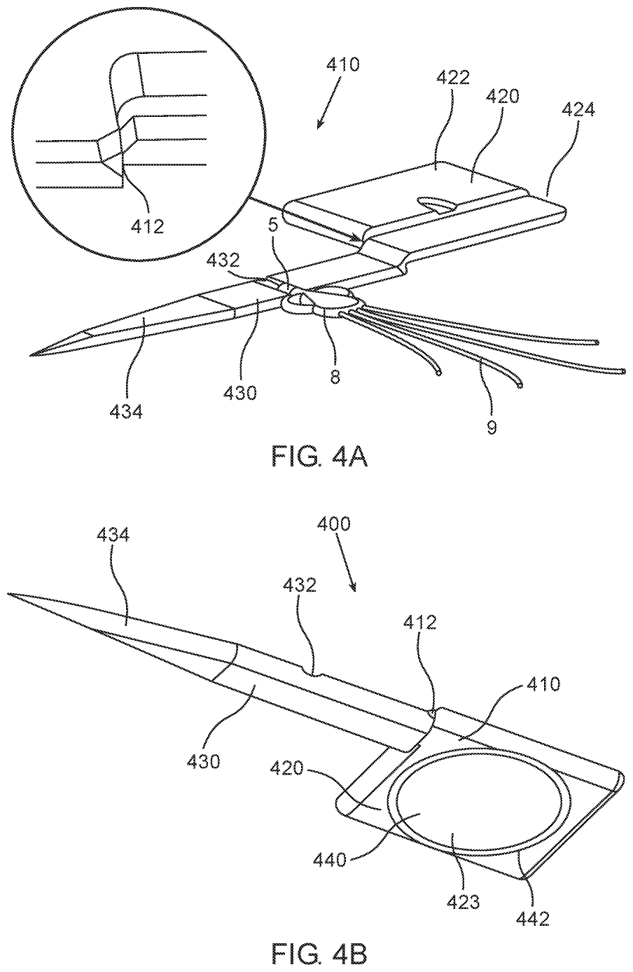

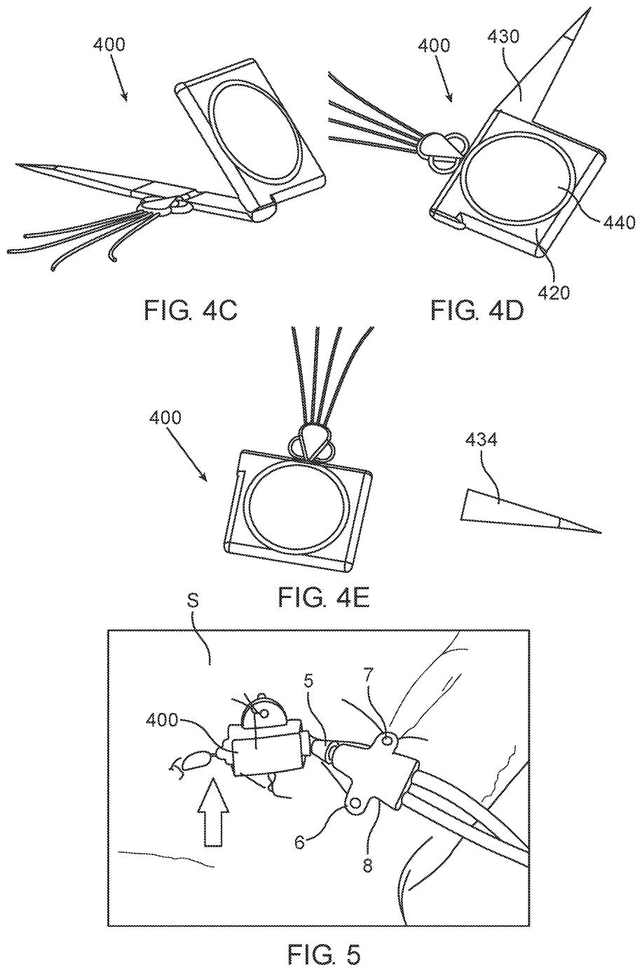

Another possible mechanism for skin stretching can include a skin stretching ring, having at least one portion comprising an adhesive surface to be attached to a skin of a subject. The skin stretching ring can be connected to two stretching opposite straps. Thus, when attached around the insertion point and pulled by the two straps, the ring can stretch the skin, thus preventing bleeding. The skin stretching ring can be similar to the elastic ring 442 shown in FIG. 4B, including an adhesive 440 on a surface configured to contact the patient's skin.

In some examples, device 100 can further include an inlet tube 30 for introducing antiseptic liquid 51 into catheter insertion cavity 12, and an outlet tube 40 for evacuating antiseptic liquid from catheter insertion cavity 12. Inlet tube 30 may be in fluid connection with a reservoir 52 of antiseptic liquid 51 (shown, for example, in FIG. 1K) to be provided continuously to catheter insertion cavity 12 and evacuated continuously from catheter insertion cavity 12, via outlet tube 40. In some examples, inlet tube 30 and outlet tube 40 may be located at two sides of catheter insertion cavity 12, as illustrated. Alternatively, inlet tube 30 and outlet tube 40 may be located elsewhere in catheter insertion cavity 12 for example, at base 10 and/or cover 20.

The evacuation of liquid from catheter insertion cavity 12 may be conducted, for example, by pumping the antiseptic liquid using a pump 50 connected to outlet tube 40, as illustrated in FIG. 1J. The pump 50 may cause sub-atmospheric pressure in catheter insertion cavity 12 causing the antiseptic liquid to flow from the reservoir into catheter insertion cavity 12 and be evacuated from catheter insertion cavity 12. Alternatively, the provision and evacuation of the antiseptic liquid may be conducted due to gravitation forces. In such case, the antiseptic liquid reservoir 52 may be held above device 100 and the exit from outlet tube 40 may be located below device 100, as illustrated in FIG. 1K.

In some examples, the continuous provision and evacuation of the antiseptic liquid 51 into and from catheter insertion cavity 12 may cause continuous flushing of the exposed exterior portion of the catheter 5 and the insertion point, thus preventing the growth of bacterial biofilm on catheter 5 and the insertion point. Furthermore, when the angle α of 30 to 90 degrees is kept between catheter 5 and the attachment surface 36, from the insertion side of catheter 5, sufficient flushing is ensured from all sides of the exposed portion of catheter 5.

Device 100 may further include a cover 20 containing antiseptic gel 22, removably connectable to base 10, configured to cover the membrane 13 and the antiseptic gel 19 of the hub chamber 11. A nonlimiting example for connectors 21 connecting base 10 to cover 20 is illustrated in FIG. 1G.

In some examples, device 100 may include one or more humidity sensors 54 attached to the base 10, optionally placed at a bottom surface of base 10, such as at attachment surface 36 (indicated, for example, in FIG. 1J), configured to detect loss of seal between the base 10 and the skin of the subject and provide an alert in response to such detection. In some examples, device 100 may include one or more pressure sensors 56 (indicated, for example, in FIG. 1J) located at the inlet tube 30 and/or the outlet tube 40, and/or inside cavity 12. When sensors, such as humidity sensors 54 or pressure sensors 56, are added, the control lines of the sensors may be combined with inlet tube 30 and/or outlet tube 40 to be connected to a control unit (not shown), that may be external to device 100, for example, hung at the bedside, along with other infusions.

The catheter 5 can extend from an end (such as a distal or front end) of a hub 8, and a plurality of tubes 9, which can be infusion tubes, can extend from another end (such as a proximal or rear end) of the hub 8. The hub chamber 11 of the base 10 is configured to accommodate the hub 8, with the catheter 9 extending from the hub 8 towards and over the membrane 13, inserted through the orifice 14 into the catheter insertion cavity 12, and through an incision in a patient's skin into the patient, for example into a vein for infusion. The hub 8 can be placed over the antiseptic gel 19 of the hub chamber 11. When the cover 20 is attached to the base 10, the hub 8 is sandwiched between the antiseptic gel 19 of the hub chamber 11 and the antiseptic gel 22 of the cover 20. The antiseptic gels 19, 22 of the base 10 and cover 20 can be compressible, allowing the hub 8 to fit tightly inside the device 100, which will affix the hub in position and prevent it from unintentionally moving or vibrating in any direction, without the need for additional stitches which are usually used to attach a hub to a patient's skin. Since the device itself is attached, optionally by an adherent attachment surface 36, to the patient's skin, this advantageously allows for easier and less traumatic affixation of the catheter 5 and hub 8 to the patient, without the need for stitching.

The device 100 can further include, in some examples, a slot 18 through which the plurality of tubes 9 can conveniently extend. The slot 18 is shown in the illustrated example to be on a rear or proximal end of the device, with the plurality of tubes 9 extending from the hub 8, through the slot 18, away from the device 100. In some examples, the base 10 can include a base slot 18A, and the cover 20 can include a cover slot 18B, such that when the cover 20 and the base 10 are attached to each other, the base slot 18A and the cover slot 18B together define the slot 18 of the device 100. Alternatively, the slot 18 can be completely defined along the body 10 without a complementary slot of the cover, or the slot 18 can be completely defined along the cover 20 without a complementary slot of the base.

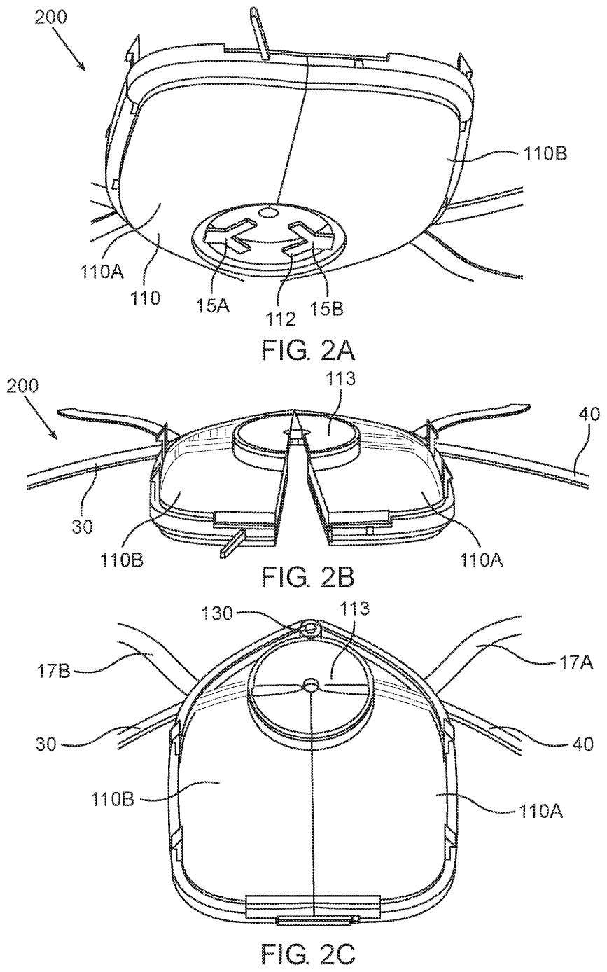

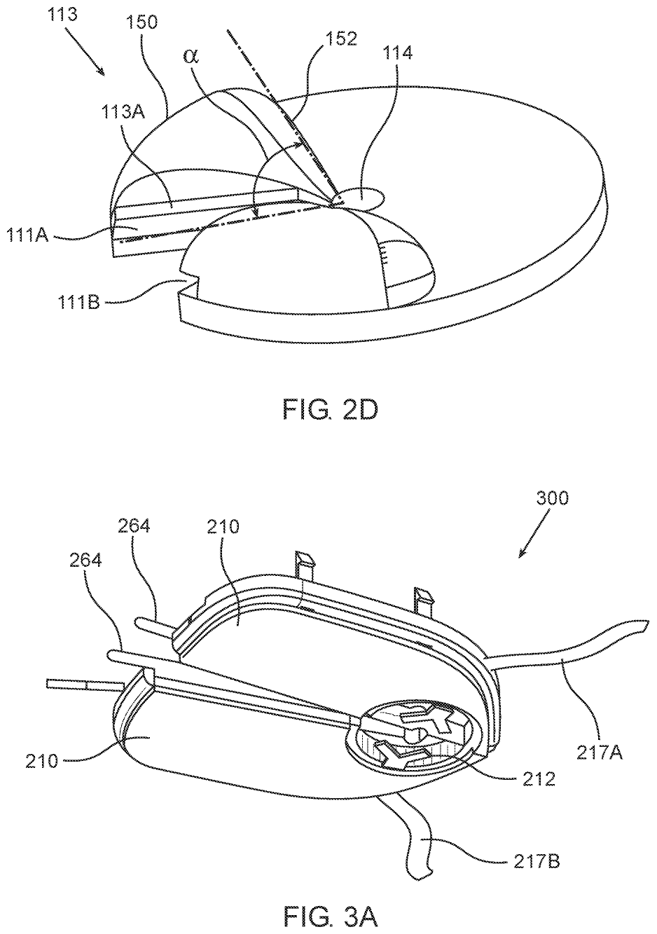

Reference is now made to FIGS. 2A, 2B, and 2C which are illustrations of a separatable antiseptic catheter fixation device according to some examples. A separatable antiseptic catheter fixation device 200 may allow separating and reattaching two parts of device 200 in order to replace device 200 without the need to replace catheter 5.

Separatable antiseptic catheter fixation device 200 can include a separatable base 110, which can be similar to any examples of base 10 described above, except that base 110 is separatable into two base parts 110A and 110B connected by a hinge 130. Each one of base parts 110A or 110B can include a portion of separatable catheter insertion cavity 112, at least one stretching element 15A or 15B, stretching strap(s) 17A and/or 17B, and either inlet tube 30 or outlet tube 40. When not separated, the catheter insertion cavity 112 can be similar to any example described herein for catheter insertion cavity 12. Device 200 can further include a detachable cover 120, which is not shown in FIGS. 2A-3C but can be similar to any example described herein for cover 20, wherein cover 120 is configured to be detached from the base 110 prior to the separation and reattaching of device 200.

In some examples, device 200 can include a membrane 113 having a catheter insertion orifice 114, illustrated in FIG. 2D, which can be generally similar to any example of membrane 13 and orifice 14 disclosed herein, except that membrane 113 comprises a partial cut 113A along a diameter of membrane 113 or a portion of a diameter of the membrane 113, crossing the catheter insertion orifice 114, allowing a partial spreading of the membrane and extraction of the catheter from catheter insertion orifice 114. In some examples, membrane 113 can include a perturbance 111A and a corresponding recess 111B at the two sides of partial cut 113A. Perturbance 111A is configured to be inserted into recess 111B to ensure the seal and completion of membrane 113.

In some examples, membrane 113 may have a bump 150, which can be in the form of a semi-curved dome and defines an angled or curved surface 152 extending from a peak of the bump 150 towards the orifice 114, wherein the angled or curved surface 152 is configured to provide support for catheter 5 along the path extending from the hub 8 towards and through the orifice 114, when inserted into orifice 114. Therefore, catheter 5 may be inserted at a sharp angle a, shown in FIG. 2D to match the same angle a described above with respect to FIG. 1G. This may optimize the insertion angle of catheter 5 at the insertion point of the patient's skin. It is to be understood that while a bump 150 defining an angled or curved surface 152 is illustrated in FIG. 2D as part of membrane 113, this is not meant to be limiting, and that a similar bump 150 with surface 152 can be added to other membranes disclosed herein, such as membrane 13 of device 100, in which case, unlike the illustrative example of FIG. 2D, the bump 150 can be a single inseparable piece.

In some examples, an antiseptic catheter fixation device may not include flushing tubes for insertion and evacuation of an antiseptic liquid from the catheter insertion cavity. In such cases, the antiseptic catheter fixation device may include antiseptic gel at both the hub chamber and the catheter insertion cavity.

Reference is now made to FIGS. 3A, 3B, 3C and 3D which are illustrations of an antiseptic catheter fixation device 300 according to some examples. An antiseptic catheter fixation device 300 includes a base 210 which can be similar to any exemplary base 10 or base 110 described herein, comprising a hub chamber 211 that includes antiseptic gel 19. In some examples, base 210 may be a separatable base, separatable into two base parts, as illustrated in FIGS. 3A-3D. In other examples, base 210 is a single inseparable piece, as illustrated with respect to base 10 of device 100.

In some examples, antiseptic catheter fixation device 300 can further comprise a catheter insertion cavity 212 located inside base 210, which includes antiseptic gel 208 therein and is covered at a first end by a membrane 213 comprising a catheter insertion orifice 214. In some examples, catheter insertion cavity 212, membrane 213 and catheter insertion orifice 214 can be substantially similar to any example of catheter insertion cavity 12, membrane 13, and catheter insertion orifice 14 disclosed herein with respect to device 100. In some examples, as illustrated in FIGS. 3A-3D, catheter insertion cavity 212, membrane 213 and catheter insertion orifice 214 can be substantially similar to any example of catheter insertion cavity 112, membrane 113 and catheter insertion orifice 114 disclosed herein with respect to device 200. In some examples, the antiseptic gel 208 inside catheter insertion cavity 212 comprises a gel orifice 239 aligned with the catheter insertion orifice 214 of the membrane 213.

In some examples, membrane 213 comprising catheter insertion orifice 214 is configured to direct the catheter to be inserted at an angle of 30 to 90 deg from the insertion side of catheter 5, as illustrated in FIG. 1G and discussed with respect to membrane 13 comprising catheter insertion orifice 14. This can be accomplished, for example, by including of a bump 150 defining an angled or curved surface 152 as described above with respect to FIG. 2D.

In some examples, antiseptic catheter fixation device 300 can further include a cover (which can be similar to any example described herein with respect to cover 20 or cover 120) that includes antiseptic gel 22, connectable to the base, configured to cover the membrane 213 and the antiseptic gel 19 of the hub chamber 211.

In some examples, antiseptic catheter fixation device 300 can include at least two (e.g., 2, 3, 4, etc.) skin stretching elements 215A and 215B that can be extracted inwards into catheter insertion cavity 212, each connect to a corresponding stretching strap 217A and 217B. The skin-stretching elements 215A, 215B and stretching straps 217A, 217B can be similar to any examples described herein for skin-stretching elements 15A, 15B and stretching straps 17A, 17B.

The antiseptic gel 208 inside catheter insertion cavity 212 can be provided with an orifice which is aligned with catheter insertion orifice 214 of the membrane 213. When the antiseptic gel 208 is provided in a separatable base, as illustrated in FIG. 3D, it can optionally include a partial cut 209 along at least a portion of a diameter thereof, which can be aligned with the partial cut of the membrane, thus allowing both to be separated simultaneously. In some examples, the antiseptic gel 208 inside catheter insertion cavity 212 can include an adhesive surface along its exposed surface facing the patient's skin (i.e., opposite to the side of the membrane 213), configured to improve attachment to the skin of the patient.

When movable skin-stretching elements 215A, 215B are included in device 300, they are positioned beneath the antiseptic gel 208 (i.e., opposite to the membrane 213), and the antiseptic gel 208 can be optionally devoid of an adhesive layer at least along the path of movement of the skin-stretching elements 215A, 215B, to allow free movement of the elements 215 and prevent them from being adhered to the antiseptic gel 208.

In some examples, the skin stretching elements can be in the form of skin stretching wires 232A and 232B, as illustrated in FIGS. 7A-7B. Each skin stretching wire 232 includes a first wire portion 234 embedded inside the antiseptic gel 208, and a second wire portion 236 extending away from the antiseptic gel 208, and optionally attached to a corresponding stretching strap 217. Each first wire portion 234 can extend along a curved path, around at least part of the circumference of the catheter insertion orifice 214, towards the partial cut 209, such that the first wire portions 234A and 234B at least partially surround the catheter insertion orifice 214 in opposite circumferential directions, each terminating at an opposite side of the antiseptic gel 208 with respect to its partial cut 209. The second wire portion 236 are continuous with the corresponding first wire portion 234, and extend out of the antiseptic gel 208 towards the thin tunnels 238A and 238B (indicated, for example, in FIG. 3D) inside the base 210. In some examples, the second wire portions 236A and 236B are attached to the corresponding stretching straps 217A and 217B, such that pulling the straps 217A and 217B away from each other tensions the corresponding skin stretching wires 232A and 232B.

Skin stretching wires 232A and 232B can be used, in some examples, instead of fork-shaped skin-stretching elements 215A and 215B, operable in a similar manner (e.g., by the user pulling on both stretching straps 217A and 217B in opposite directions) to stretch the subject's skin. FIG. 3E shows the skin stretching wires 232A and 232B in a free untensioned state, wherein the antiseptic gel 208 can be optionally split with both sides of the partial cut 209 spaced from each other. When the second wire portions 236A and 236B are pulled sideways in opposite directions, optionally by pulling stretching straps 217A and 217B attached thereto, the skin stretching wires 232A and 232B are tensioned in a manner that reduces their radii of curvature around the orifice 214, thereby approximating both sides of the partial cut 209 towards each other while optionally also reducing the size of the orifice 214, which can tighten is around the catheter 5. The antiseptic gel 208 can have an adherent lower surface 237 to facilitate adherence to the patient's skin, such that tightening the wires 232A and 232B to move both parts of the antiseptic gel 208 one both sides of the partial cut 209 towards each other also serves to stretch the patient's skin. In some examples, after the straps 217A and 217B are pulled to tension the wires 232A and 232B, the straps 217 can be affixed in position by any suitable manner (such as inclusion of buckles or other clamping solutions) to maintain them in this state, which will also forcibly press both sides of the antiseptic gel 208 to improve sealing of the partial cut 209.

It is to be understood that while skin stretching wires 232A and 232B are illustrated in FIGS. 3E-3F and described herein with respect to a separable antiseptic gel 208 that includes a partial cut 209, the skin stretching wires 232A and 232B can be similarly utilized, in some examples, with an antiseptic gel 208 of the catheter insertion cavity, which is not necessarily separable and may be devoid of a partial cut 209.

Another possible mechanism of device 300 for skin stretching can include a skin stretching ring, having at least one portion comprising an adhesive surface to be attached to a skin of a subject. The skin stretching ring can be connected to two stretching opposite straps. Thus, when attached around the insertion point and pulled by the two straps, the ring can stretch the skin, thus preventing bleeding. The skin stretching ring of device 300 can be similar to the elastic ring 442 shown in FIG. 4B, including an adhesive material 440 on a surface configured to contact the patient's skin.

In some examples, a separatable base such as any of base 210 or 110, can be separated into two part that form two inner separatable sides 266 facing each other, as shown in FIGS. 3A, 3B and 3D. any of the sides 266 can be part of not only the rigid material from which the base 210 or 110 is formed, but also part of the antiseptic gel 19 that can be contained in the corresponding hub chamber, the antiseptic gel 19 also configured to separate into two portions along with the separated part of the base. In some example, one or both inner separatable sides 266 can include an adhesive layer, which can be covered by a protective film 262. The protective film 262 can cover the adhesive layer of the corresponding side 266, and can optionally include a tab 264 that can be grabbed by a user to peel away the protective film 262, thereby exposing the adhesive layer, such that when the sides 266 are approximated to each other, adherence therebetween can retain the device 200 or 300 in a closed (i.e., not separated) configuration.

Accordingly, devices 100, 200 and/or 300 can provide, central catheter sterilization, catheter fixation, and bleeding prevention.

Some additional aspects of the invention may be directed to a foldable antiseptic dressing configured to provide central catheter sterilization using a simple one-piece flexible pad made primarily from antiseptic gel. In such case, the fixation of the catheter is achieved by stitching two fixation arms included in the catheter to the patient. A foldable antiseptic dressing according to examples of the invention may allow replacing the dressing without the need to replace the stitching of the hub 8 or the catheter 5.

Reference is now made to FIGS. 4A, 4B, 4C, 4D, and 4E which are illustrations of a foldable antiseptic dressing according to some examples. A foldable antiseptic dressing 400 can include a foldable pad 410 made from an antiseptic gel, and an adhesive material 440. Foldable pad 410 is primarily or entirely made from an antiseptic gel. Such an antiseptic gel can have the required mechanical strength and flexibility for supporting and fixating a central catheter in the central insertion point.

In some examples, foldable pad 410 can include a folding portion 420 comprising a thick support 422 and a thinner strap 424. Foldable pad 410 can further include a catheter insertion strap 430 comprising a recess 432 for holding a catheter 5. In some examples, catheter insertion strap 430 is pivotally connected to thinner strap 424. In some examples, the thickness of catheter insertion strap 430 is equal to or higher than the difference between the thicknesses of thick support 422 and thinner strap 424, such that upon folding portion 420 on top of catheter insertion strap 430, as illustrated in FIGS. 4C-4D, catheter insertion strap 430 fully fills the “step” between thick support 422 and thinner strap 424 (shown in FIG. 4D).

In some examples, in the folded state, illustrated in FIG. 4E, catheter 5 is located between catheter insertion strap 430 and thinner strap 424. Following the folding, an end portion 434 of catheter insertion strap 430 may be detached from antiseptic dressing 400, leaving only the foldable portion to encompass catheter 5.

In some examples, foldable pad 410 is made of a single piece, and folding portion 420 is connected to catheter insertion strap 430 via a film 412 made from the antiseptic gel, as shown in the enlarged illustration circled in FIG. 4A. In such cases, foldable pad 410 may be cast into a mold, injected molded, printed using 3D printer, or may be fabricated using any suitable method.

In some examples, folding portion 420 is connected to catheter insertion strap 430 via a pivotable connector (not illustrated) which is not necessarily integrally formed with the folding portion 420 and/or catheter insertion strap 430. In such cases, each one of folding portion 420 and catheter insertion strap 430 can be fabricated separately from antiseptic gel (e.g., using casting, injection molding, printing, etc.), and then may be connected to each other via a hinge or an axis allowing folding portion 420 to be folded on top of catheter insertion strap 430.

The adhesive material 440 is configured to fixate the foldable antiseptic dressing 400 to a skin of a subject, such that the recess 432 faces away from a skin of the subject when adhered thereto. In some examples, adhesive material 440 comprises at least one of: an adhesive layer coating at least a portion of an attachment surface 423 of the thick support 422, and an adhesive sticker attached to at least a portion of the attachment surface 423 of the thick support 422. In some examples, pad 410 may include an elastic ring 442 (e.g., a metallic elastic ring) embedded in the pad, which is configured to stretch the skin of the patient, during the attachment, in order to avoid bleeding from the insertion point. Pressing the metallic elastic ring 442 from its sides towards its center during the attachment of pad 410 to the skin may form a stretching effect on the patient's skin, when released, as the ring is allowed to resiliently return to its unpressed or uncompressed free-state configuration. As should be understood by one skilled in the art, the elastic ring 442 illustrated in FIGS. 4B-4E is given as an example only. Therefore, elastic ring 442 can have any geometrical shape that can ensure the ability of stretch the skin of the patient, during the attachment.

Reference in now made to FIG. 5 which is an image of a foldable antiseptic dressing 400 encompassing a catheter 5 fixed by stitching to a skin S of a patient. Two fixation arms 6 extending from the hub 8 can be stitched by stitches 7 to the skin S of the patient. In some examples, foldable antiseptic dressing 400 can be unfolded and opened in order to be removed and replaced without the need to open stitches 7 or replace catheter 5. A new foldable antiseptic dressing 400 can be then provided in the unfolded state (as illustrated in FIGS. 4A and 4B) and placed such that catheter 5 is inserted in recess 432 followed by folding the foldable portion 420, thereby providing the required antiseptic protection to the catheter 5 and the insertion region at the patient's skin S.

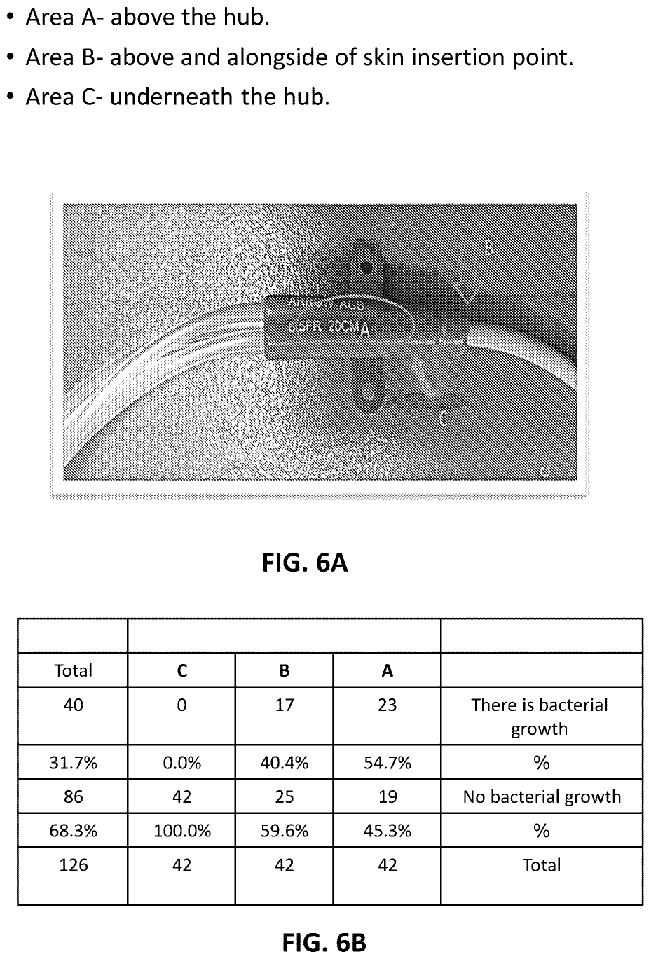

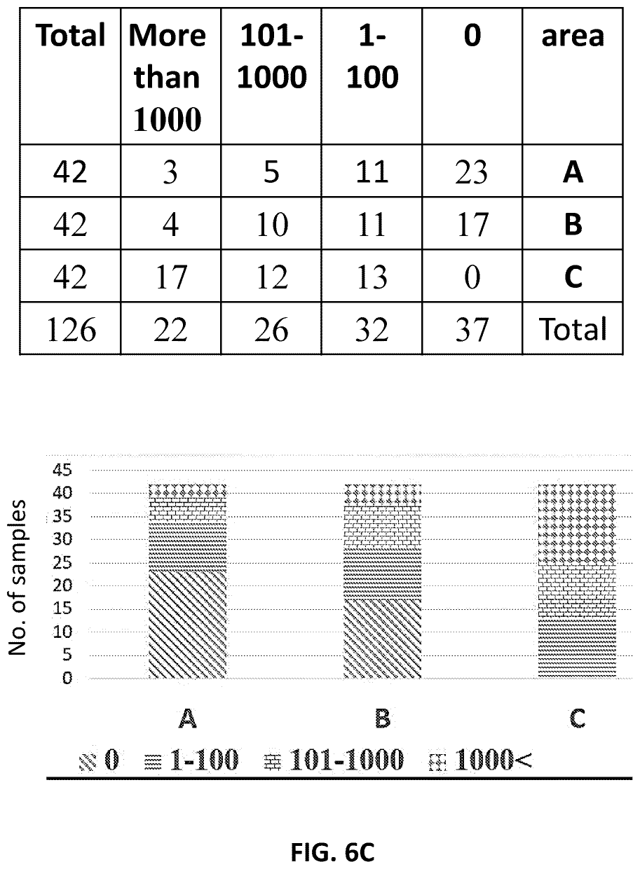

Reference is now made to FIGS. 6A, 6B, and 6C, which show an image, tables, and a graph summarizing an experiment that demonstrated the increased rate of bacteria/yeasts growth under a hub of a central catheter that can be used with the devices and dressing disclosed herein. As shown in FIGS. 6A-6C, the area under the hub of the catheter and alongside skin insertion point are the most sensitive to an increased bacteria/yeast growth rate. The disclosed results show quantitatively the necessity of providing continuous antiseptic protection to a central catheter, for example, the antiseptic protection provided by any one of the antiseptic catheter fixation devices 100, 200, 300, or by foldable antiseptic dressing 400 discussed herein above.

### Some Examples of the Disclosed Implementations

Some examples of above-described implementations are enumerated below. It should be noted that one feature of an example in isolation or more than one feature of the example taken in combination and, optionally, in combination with one or more features of one or more examples below are examples also falling within the disclosure of this application.

### Example 1

An antiseptic catheter fixation device, comprising:
a base comprising:
   a hub chamber;
   antiseptic gel inside the hub chamber;
   a catheter insertion cavity surrounded by a cavity sidewall;
   a membrane covering a first end of the catheter insertion cavity, the membrane comprising a catheter insertion orifice; and
a cover removably connectable to the base, the cover comprising antiseptic gel.

### Example 2

The device of any example herein, particularly of example 1, wherein the base comprises an attachment surface opposite to the side of the hub chamber.

### Example 3

The device of any example herein, particularly of example 2, wherein the attachment surface comprises an adhesive surface configured to be attached to a skin of a subject.

### Example 4

The device of any example herein, particularly of any one of examples 1 to 3, wherein the membrane is configured to direct a catheter to be inserted through the catheter insertion cavity at an angle of 30 to 90 degrees from an insertion side of a catheter, when the catheter is inserted into the catheter insertion orifice.

### Example 5

The device of any example herein, particularly of any one of examples 1 to 4, wherein the membrane comprises a bump defining an angled surface extending towards the catheter insertion cavity.

### Example 6

The device of any example herein, particularly of example claim 5, wherein the bump is in the form of a semi-curved dome.

### Example 7

The device of any example herein, particularly of any one of examples 1 to 6, further comprising a slot at a proximal end of the antiseptic catheter fixation device, configured to allow passage of a plurality of tubes extending from a hub therethrough, when the hub resides inside the hub chamber.

### Example 8

The device of any example herein, particularly of any one of examples 1 to 7, wherein the antiseptic gel inside the hub chamber and the antiseptic gel inside the cover are compressible, configured to accommodate a hub of a catheter therebetween.

### Example 9

The device of any example herein, particularly of any one of examples 1 to 8, wherein the base is separatable into two parts that form two inner separatable sides facing each other.

### Example 10

The device of any example herein, particularly of example 9, wherein the membrane comprises a partial cut extending from the catheter insertion cavity, and configured to allow partial spreading of the membrane.

### Example 11

The device of any example herein, particularly of example 10, wherein the membrane further comprises a perturbance and a corresponding recess at two sides of the partial cut of the membrane.

### Example 12

The device of any example herein, particularly of any one of examples 9 to 11, wherein at least one of the two inner separatable sides comprises an adhesive layer covered by a removable protective film.

### Example 13

The device of any example herein, particularly of any one of examples 1 to 12, further comprising antiseptic gel inside the catheter insertion cavity.

### Example 14

The device of any example herein, particularly of example 13, wherein the antiseptic gel inside the catheter insertion cavity comprises an adherent lower surface.

### Example 15

The device of any example herein, particularly of any one of examples 13 or 14, wherein the antiseptic gel inside the catheter insertion cavity comprises a gel orifice which is aligned with the catheter insertion orifice of the membrane.

### Example 16

The device of any example herein, particularly of any one of examples 1 to 15, further comprising at least two skin stretching elements extracted inwards into the catheter insertion cavity, wherein each of skin stretching element is attached to a stretching strap.

### Example 17

The device of any example herein, particularly of example 16, wherein each skin stretching element comprises an adhesive surface configured to be attached to a skin of a subject.

### Example 18

The device of any example herein, particularly of any one of examples 16 or 17, wherein each skin stretching element is configured to move within the catheter insertion cavity.

### Example 19

The device of any example herein, particularly of any one of examples 16 to 18, wherein each skin stretching element comprises a V-shaped end portion.

### Example 20

The device of any example herein, particularly of example 15, further comprises at least two skin stretching wires, wherein each skin stretching wire comprises a first wire portion embedded in the antiseptic gel which is inside the catheter insertion cavity, and a second wire portion extending out of the antiseptic gel which is inside the catheter insertion cavity.

### Example 21

The device of any example herein, particularly of example 20, wherein each first wire portion at least partially surround the gel orifice.

### Example 22

The device of any example herein, particularly of any one of examples 20 or 21, wherein each the at least two first wire portions extend around the gel orifice in opposite directions.

Example 23. The device of any one of any example herein, particularly of any one of examples 20 to 22, wherein each second wire portion is attached to a stretching strap.

### Example 24

The device of any example herein, particularly of any one of examples 1 to 12, further comprises:
an inlet tube in fluid communication with the catheter insertion cavity, configured to introduce antiseptic liquid into the catheter insertion cavity; and
an outlet tube in fluid communication with the catheter insertion cavity, configured to evacuate antiseptic liquid from the catheter insertion cavity.

### Example 25

The device of any example herein, particularly of example 24, further comprising a pump in fluid connection with the outlet tube, configured to pump the antiseptic liquid from the catheter insertion cavity.

### Example 26

The device of any example herein, particularly of example 24, further comprising an antiseptic liquid reservoir in fluid connection to the inlet tube, located above the antiseptic catheter fixation device, wherein an exit of the outlet tube is located below the antiseptic catheter fixation device.

### Example 27

The device of any example herein, particularly of any one of examples 24 to 26, further comprising at least one pressure sensor located inside at least one of: the inlet tube, the outlet tube, and the catheter insertion cavity.

Example 28. The device of any example herein, particularly of any one of examples 1 to 27, further comprising at least one humidity sensor attached to the base, configured to detect loss of seal between the base and a skin of a subject.

### Example 29 A foldable antiseptic dressing, comprising:

a foldable pad made from an antiseptic gel, comprising:
   a folding portion comprising a thick support and a thinner strap; and
   a catheter insertion strap comprising a recess for holding a catheter, pivotally connected to the thinner strap; and
an adhesive material covering at least a portion of an attachment surface of the thick support, such that at a folded state, the adhesive material is configured to fixate the foldable antiseptic catheter fixation pad to a skin of a subject, such that the recess faces away from the skin of the subject when adhered thereto.

### Example 30

The dressing of any example herein, particularly of examples 29, wherein the thickness of the catheter insertion strap is equal to or higher than a difference between the thicknesses of the thick support and the thinner strap.

### Example 31

The dressing of any example herein, particularly of any one of examples 29 or 30, wherein the foldable pad is made of a single piece and the folding portion is connected to the catheter insertion strap via a film made from the antiseptic gel.

### Example 32

The foldable antiseptic catheter dressing of any example herein, particularly of any one of examples 29 or 30, wherein the folding portion is connected to the catheter insertion strap via an additional connector.

### Example 33

The dressing of any example herein, particularly of any one of examples 29 to 32, wherein the adhesive material is at least one of: an adhesive layer coating at least the portion of the attachment surface of the thick support, and an adhesive sticker attached to at least the portion of the attachment surface of the thick support.

### Example 34

The dressing of any example herein, particularly of any one of examples 29 to 33, further comprising an elastic ring embedded in the pad.

While certain features of the invention have been illustrated and described herein, many modifications, substitutions, changes, and equivalents may occur to those skilled in the art. It is, therefore, to be understood that the appended claims are intended to cover all such modifications and changes as fall within the true spirit of the invention.

1. An antiseptic catheter fixation device, comprising:
a base comprising:
a hub chamber;
antiseptic gel inside the hub chamber;
a catheter insertion cavity surrounded by a cavity sidewall;
a membrane covering a first end of the catheter insertion cavity, the membrane comprising a catheter insertion orifice; and
a cover removably connectable to the base, the cover comprising antiseptic gel.

2. The device of claim 1, wherein the base comprises an attachment surface opposite to the side of the hub chamber.

3. The device of claim 2, wherein the attachment surface comprises an adhesive surface configured to be attached to a skin of a subject.

4. The device of claim 1, wherein the membrane is configured to direct a catheter to be inserted through the catheter insertion cavity at an angle of 30 to 90 degrees from an insertion side of a catheter, when the catheter is inserted into the catheter insertion orifice.

5. The device of claim 1, wherein the membrane comprises a bump defining an angled surface extending towards the catheter insertion cavity.

6. The device of claim 5, wherein the bump is in the form of a semi-curved dome.

7. The device of claim 1, further comprising a slot at a proximal end of the antiseptic catheter fixation device, configured to allow passage of a plurality of tubes extending from a hub therethrough, when the hub resides inside the hub chamber.

8. The device of claim 1, wherein the antiseptic gel inside the hub chamber and the antiseptic gel inside the cover are compressible, configured to accommodate a hub of a catheter therebetween.

9. The device of claim 1, wherein the base is separatable into two parts that form two inner separatable sides facing each other.

10. The device of claim 9, wherein the membrane comprises a partial cut extending from the catheter insertion cavity, and configured to allow partial spreading of the membrane.

11. The device of claim 10, wherein the membrane further comprises a perturbance and a corresponding recess at two sides of the partial cut of the membrane.

12. The device of claim 9, wherein at least one of the two inner separatable sides comprises an adhesive layer covered by a removable protective film.

13. The device of claim 1, further comprising antiseptic gel inside the catheter insertion cavity.

14. The device of claim 13, wherein the antiseptic gel inside the catheter insertion cavity comprises an adherent lower surface.

15. The device of claim 13, wherein the antiseptic gel inside the catheter insertion cavity comprises a gel orifice which is aligned with the catheter insertion orifice of the membrane.

16. The device of claim 1, further comprising at least two skin stretching elements extracted inwards into the catheter insertion cavity, wherein each of skin stretching element is attached to a stretching strap.

17-19. (canceled)

20. The device of claim 15, further comprises at least two skin stretching wires, wherein each skin stretching wire comprises a first wire portion embedded in the antiseptic gel which is inside the catheter insertion cavity, and a second wire portion extending out of the antiseptic gel which is inside the catheter insertion cavity.

21-23. (canceled)

24. The device of claim 1, further comprises:
an inlet tube in fluid communication with the catheter insertion cavity, configured to introduce antiseptic liquid into the catheter insertion cavity; and
an outlet tube in fluid communication with the catheter insertion cavity, configured to evacuate antiseptic liquid from the catheter insertion cavity.

25. The device of claim 24, further comprising a pump in fluid connection with the outlet tube, configured to pump the antiseptic liquid from the catheter insertion cavity.

26-27. (canceled)

28. The device of claim 1, further comprising at least one humidity sensor attached to the base, configured to detect loss of seal between the base and a skin of a subject.

29-34. (canceled)
